(19) United States
(12) Patent Application Publication
(10) Pub. No.: US 2006/0074080 A1
(43) Pub. Date: Apr. 6, 2006

(54) Combination product comprising melagatran and an anti-arrhythmic oxabispidenes

(75) Inventor: Elisabeth Svernhage, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/513,187

(22) PCT Filed: May 5, 2003
(86) PCT No.: PCT/SE03/00720

§ 371 Date: Nov. 1, 2004

(30) Foreign Application Priority Data
May 6, 2002 (SE) 0201373-8

(51) Int. Cl. A61K 31/5383; A61K 31/47

(52) U.S. Cl. 514/230.5; 514/310

(57) ABSTRACT

There is provided a combination product comprising: (1) melagatran or a pharmaceutically-acceptable derivative thereof; and (1) a compound as defined in claim 1 of WO 01/28992 or (2) a compound of Claim 34 of WO 01/28992 or (3) Compound A or B or C or D (or pharmaceutically-acceptable salts thereof) for use in treating arrhythmia or a coagulation controlled complication thereof.

### FIELD OF THE INVENTION

This invention relates to a new combination of pharmaceutically-active compounds. In particular the invention relates to a combination of melagatran or a pharmaceutically-acceptable derivative thereof and certain antiarrhythmic oxabispidines or pharmaceutically acceptable salts thereof.

### BACKGROUND TO THE INVENTION

Atrial fibrillation (AF) is characterised by grossly disorganised atrial electrical activity that is irregular in respect of both rate and rhythm. Patients with AF have no visually discernible timing pattern in atrial electrical activity when measured by surface ECG, or in electrogram sequences recorded by catheter electrodes.

During AF, the regular pumping action of the atria is replaced by irregular, disorganised and quivering spasms of atrial tissue. These spasms may be experienced as irregular heartbeat, palpitations, discomfort, dizziness and/or angina pectoris. Further, the inefficient pumping action of the heart tends to lead to significant morbidity related to reduced blood flow. More seriously, the reduced cardiac output can lead to blood pooling in the left atria and the formation of blood clots. Blood clots, mostly originating in the left atrium, can dislodge and travel through the bloodstream to organs, e.g. the brain, spleen, kidneys etc. If the clot travels to the brain, this may result in cerebral stroke and even death.

In the US alone, AF affects an estimated two million people, with approximately 160,000 new cases being diagnosed each year. It has been estimated that AF is responsible for over 70,000 strokes each year in the US, and that the cost of treating these patients is more than US$3.6 billion annually. The cost of drug treatment for AF alone has been estimated to be in excess of US$400 million world-wide each year.

AF can be classified in two broadly defined groups: “valvular” AF and “non-valvular” AF (NVAF). In valvular AF, the arrhythmia is experienced due to a disorder of one or more of the heart valves (e.g. valvular disease), or the presence of mechanical (prosthetic) heart valves. Conversely, NVAF is AF experienced in the case where there is an absence of significant valvular disease or prosthesis.

The oxabispidine compounds of international patent application WO 01/28992 are indicated as being useful in the treatment of cardiac arrhythmias. WO 01/28992 is incorporated herein by reference. Claim 1 of WO 01/28992 reads:

A compound of formula I, 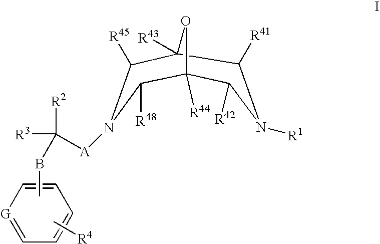
wherein

R¹ represents C₁₋₁₂ alkyl (which alkyl group is optionally substituted and/or terminated by one or more groups selected from halo, cyano, nitro, aryl, Het¹, —C(O)R^{5a}, —OR^{5b}, —N(R⁶)R^{5c}, —C(O)XR⁷, —C(O)N(R⁸)R^{5d}, and —S(O)₂R⁹), or R¹ represents —C(O)XR⁷, —C(O)N(R⁸)R^{5d} or —S(O)₂R⁹;

R^{5a} to R^{5d} independently represent, at each occurrence, H, C₁₋₆ alkyl (which latter group is optionally substituted and/or terminated by one or more substituents selected from —OH, halo, cyano, nitro, aryl and Het²), aryl or Het³, or R^{5d}, together with R⁸, represents C₃₋₆ alkylene (which alkylene group is optionally interrupted by an O atom and/or is optionally substituted by one or more C₁₋₃ alkyl groups);

R⁶ represents H, C₁₋₆ alkyl (optionally substituted and/or terminated by one or more substituents selected from —OH, halo, cyano, nitro and aryl), aryl, —C(O)R^{10a}, —C(O)OR^{10b} or —C(O)N(H)R^{10c};

R^{10a}, R^{10b} and R^{10c} independently represent C₁₋₆ alkyl (optionally substituted and/or terminated by one or more substituents selected from —OH, halo, cyano, nitro and aryl), aryl, or R^{10a} represents H;

R⁷ represents C₁₋₁₂ alkyl (optionally substituted and/or terminated by one or more substituents selected from —OH, halo, cyano, nitro, aryl,

C₁₋₆ alkoxy and Het⁴);

R⁸ represents H, C₁₋₁₂ alkyl, C₁₋₆ alkoxy (which latter two groups are optionally substituted and/or terminated by one or more substituents selected from —OH, halo, cyano, nitro, C₁₋₄ alkyl and C₁₋₄ alkoxy), -D-aryl, -D-aryloxy, -D-Het⁵, -D-N(H)C(O)R^{11a}, -D-S(O)₂R^{12a}, -D-C(O)R^{11b}, -D-C(O)OR^{12b}, -D-C(O)N(R^{11c})R^{11d}, or R⁸, together with R^{5d}, represents C₃₋₆ alkylene (which alkylene group is optionally interrupted by an O atom and/or is optionally substituted by one or more C₁₋₃ alkyl groups);

R^{11a} to R^{11d} independently represent H, C₁₋₆ alkyl (optionally substituted and/or terminated by one or more substituents selected from —OH, halo, cyano, nitro and aryl), aryl, or R^{11c} and R^{11d} together represent C₃₋₆ alkylene;

R⁹, R^{12a} and R^{12b} independently represent C₁₋₆ alkyl (optionally substituted and/or terminated by one or more substituents selected from —OH, halo, cyano, nitro and aryl) or aryl;

D represents a direct bond or C₁₋₆ alkylene;

X represents O or S;

R² represents H, halo, C₁₋₆ alkyl, —OR¹³, -E-N(R¹⁴)R¹⁵ or, together with R³, represents ═O;

R³ represents H, C₁₋₆ alkyl or, together with R², represents ═O;

R¹³ represents H, C₁₋₆ alkyl, -E-aryl, -E-Het⁶, —C(O)R^{16a}, —C(O)OR^{16b} or —C(O)N(R^{17a})R^{17b};

R¹⁴ represents H, C₁₋₆ alkyl, -E-aryl, -E-Het⁶, —C(O)R^{16a}, —C(O)OR^{16b}, —S(O)₂R^{16c}, —[C(O)]ₚN(R^{17a})R^{17b} or —C(NH)NH₂;

R¹⁵ represents H, C₁₋₆ alkyl, -E-aryl or —C(O)R^{16d};

R^{16a} to R^{16d} independently represent, at each occurrence when used herein, C₁₋₆ alkyl (optionally substituted and/or terminated by one or more substituents selected from halo, aryl and Het⁷), aryl, Het⁸, or R^{16a} and R^{16d} independently represent H;

R^{17a} and R^{17b} independently represent, at each occurrence when used herein, H or C₁₋₆ alkyl (optionally substituted and/or terminated by one or more substituents selected from halo, aryl and Het⁹), aryl, Het¹⁰, or together represent C₃₋₆ alkylene, optionally interrupted by an O atom;

E represents, at each occurrence when used herein, a direct bond or C₁₋₄ alkylene;

p represents 1 or 2;

Het¹ to Het¹⁰ independently represent five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{18a})R^{18b}, —C(O)R^{18c}, —C(O)OR^{18d}, —C(O)N(R^{18e})R^{18f}, —N(R^{18g})C(O)R^{18h} and —N(R¹⁸ⁱ)S(O)₂R^{18j};

R^{18a} to R^{18j} independently represent C₁₋₆ alkyl, aryl or R^{18a} to R¹⁸ⁱ independently represent H;

A represents a direct bond, -J-, -J-N(R¹⁹)- or -J-O— (in which latter two groups, N(R¹⁹)- or O— is attached to the carbon atom bearing R² and R³);

B represents -Z-, -Z-N(R²⁰)-, —N(R²⁰)-Z-, -Z-S(O)ₙ-, -Z-O— (in which latter two groups, Z is attached to the carbon atom bearing R² and R³), —N(R²⁰)C(O)O-Z-, (in which latter group, —N(R²⁰) is attached to the carbon atom bearing R² and R³) or —C(O)N(R²⁰)- (in which latter group, —C(O) is attached to the carbon atom bearing R² and R³);

J represents C₁₋₆ alkylene optionally substituted by one or more substituents selected from —OH, halo and amino;

Z represents a direct bond or C₁₋₄ alkylene;

n represents 0, 1 or 2;

R¹⁹ and R²⁰ independently represent H or C₁₋₆ alkyl;

G represents CH or N;

R⁴ represents one or more optional substituents selected from —OH, cyano, halo, nitro, C₁₋₆ alkyl (optionally terminated by —N(H)C(O)OR^{21a}), C₁₋₆ alkoxy, N(R^{22a})R^{22b}, —C(O)R^{22c}, C(O)OR^{22d}, —C(O)N(R^{22e})R^{22f}, —N(R^{22g})C(O)R^{22h}, —N(R²²ⁱ)C(O)N(R^{22j})R^{22k}, —N(R^{22m})S(O)₂R^{21b}, —S(O)₂R^{21c}, and/or —OS(O)₂R^{21d};

R^{21a} to R^{21d} independently represent C₁₋₆ alkyl;

R^{22a} and R^{22b} independently represent H, C₁₋₆ alkyl or together represent C₃₋₆ alkylene, resulting in a four- to seven-membered nitrogen-containing ring;

R^{22c} to R^{22m} independently represent H or C₁₋₆ alkyl; and

R⁴¹ to R⁴⁶ independently represent H or C₁₋₃ alkyl;

wherein each aryl and aryloxy group, unless otherwise specified, is optionally substituted;

provided that

(a) the compound is not:
3,7-dibenzoyl-9-oxa-3,7-diazabicyclo[3.3.1]nonane;

(b) when A represents -J-N(R¹⁹)- or -J-O—, then:
(i) J does not represent C₁ alkylene; and
(ii) B does not represent —N(R²⁰)-, —N(R²⁰)-Z- (in which latter group N(R²⁰) is attached to the carbon atom bearing R² and R³), —S(O)ₙ-, —O— or —N(R²⁰)C(O)O-Z- when R² and R³ do not together represent ═O; and

(c) when R² represents —OR¹³ or —N(R¹⁴)(R¹⁵), then:
(i) A does not represent -J-N(R¹⁹)- or -J-O-; and
(ii) B does not represent —N(R²⁰)-, —N(R²⁰)-Z- (in which latter group N(R²⁰) is attached to the carbon atom bearing R² and R³), —S(O)ₙ-, —O— or —N(R²⁰)C(O)O-Z-;
   or a pharmaceutically acceptable derivative thereof.

This definition will hereinafter be referred to as a compound as defined in claim 1 of WO 01/28992. The definition of “a pharmaceutically acceptable derivative thereof” is that used in WO 01/28992 which is now repeated. Pharmaceutically acceptable derivatives include salts and solvates. Salts which may be mentioned include acid addition salts. Specific salts that may be mentioned include arylsulfonate salts, such as toluenesulfonate and, especially, benzenesulfonate salts. Solvates that may be mentioned include hydrates, such as monohydrates of the compounds of the invention.

Pharmaceutically acceptable derivatives also include, at the oxabispidine or (when G represents N) pyridyl nitrogens, C₁₋₄ alkyl quaternary ammonium salts and N-oxides, provided that when a N-oxide is present:

no Het (Het¹, Het², Het³, Het⁴, Het⁵, Het⁶, Het⁷, Het⁸, Het⁹ and Het¹⁰) group contains an unoxidised S-atom; and/or

n does not represent O when B represents -Z-S(O)ₙ-.

The compounds of the invention may exhibit tautomerism. All tautomeric forms and mixtures thereof are included within the scope of the invention.

Claim 34 of WO 01/28992 provides a list of compounds as follows

A compound which is:
4-{2-[7-(3,3-dimethyl-2-oxobutyl)-9-oxa-3,7-diazabicyclo[3.3.1]non-3-yl]ethyl}benzonitrile;
7-[4-(4-cyanophenyl)-4-(3,4-dimethoxyphenoxy)butyl]-N-ethyl-9-oxa-3,7-diazabicyclo[3.3.1]nonane-3-carboxamide;
4-({3-[7-(3,3-dimethyl-2-oxobutyl)-9-oxa-3,7-diazabicyclo[3.3.1]non-3-yl]propyl}amino)benzonitrile;
4-{3-[7-(4-fluorobenzyl)-9-oxa-3,7-diazabicyclo[3.3.1]non-3-yl]-2-hydroxypropoxy}benzonitrile;
4-(2-{7-[2-(4-methoxyphenyl)-2-oxoethyl]-9-oxa-3,7-diazabicyclo[3.3.1]non-3-yl}ethoxy)benzonitrile;
4-[((2S)-2-amino-3-{7-[2-(1H-pyrrol-1-yl)ethyl]-9-oxa-3,7-diazabicyclo[3.3.1]non-3-yl}propyl)oxy]benzonitrile;
tert-butyl 2-{7-[3-(4-cyanoanilino)propyl]-9-oxa-3,7-diazabicyclo[3.3.1]-non-3-yl}ethylcarbamate;
tert-butyl 2-{7-[4(4-cyanophenyl)butyl]-9-oxa-3,7-diazabicyclo[3.3.1]-non-3-yl}ethylcarbamate;
tert-butyl 2-{7-[(2S)-3-(4-cyanophenoxy)-2-hydroxypropyl]-9-oxa-3,7-diazabicyclo[3.3.1]non-3-yl}ethylcarbamate;
4-(2-{7-[4-(4-pyridinyl)butyl]-9-oxa-3,7-diazabicyclo[3.3.1]non-3-yl}-ethoxy)benzonitrile tert-butyl 2-{7-[4-(4-pyridinyl)butyl]-9-oxa-3,7-diazabicyclo[3.3.1]non-3-yl}ethylcarbamate;
4-{3-[7-(3,3-dimethyl-2-oxobutyl)-9-oxa-3,7-diazabicyclo[3.3.1]non-3-yl]-2-hydroxypropoxy}benzonitrile;
4-{3-[7-(3,4-dimethoxyphenethyl)-9-oxa-3,7-diazabicyclo[3.3.1]non-3-yl]-2-hydroxypropoxy}benzonitrile;
4-{2-[7-(3,3-dimethyl-2-oxobutyl)-9-oxa-3,7-diazabicyclo[3.3.1]non-3-yl]-ethoxy}benzonitrile;
4-({3-[7-(butylsulfonyl)-9-oxa-3,7-diazabicyclo[3.3.1]non-3-yl]propyl}-amino)benzonitrile;
4-({3-[7-(3,4-dimethoxyphenethyl)-9-oxa-3,7-diazabicyclo[3.3.1]non-3-yl]propyl}amino)benzonitrile;
4-[4-[7-(butylsulfonyl)-9-oxa-3,7-diazabicyclo[3.3.1]non-3-yl]-1-(3,4-dimethoxyphenoxy)butyl]benzonitrile;
4-{1-(3,4-dimethoxyphenoxy)-4-[7-(3,3-dimethyl-2-oxobutyl)-9-oxa-3,7-diazabicyclo[3.3.1]non-3-yl]butyl}benzonitrile;
4-[4-[7-(3,4-dimethoxyphenethyl)-9-oxa-3,7-diazabicyclo[3.3.1]non-3-yl]-1-(3,4-dimethoxyphenoxy)butyl]benzonitrile;
2-(4-acetyl-1-piperazinyl)ethyl 7-[3-(4-cyanophenoxy)-2-hydroxypropyl]-9-oxa-3,7-diazabicyclo[3.3.1]nonane-3-carboxylate;
7-[3-(4-cyanophenoxy)-2-hydroxypropyl]-N-ethyl-9-oxa-3,7-diazabicyclo[3.3.1]nonane-3-carboxamide;
4-{3-[7-(butylsulfonyl)-9-oxa-3,7-diazabicyclo[3.3.1]non-3-yl]-2-hydroxypropoxy}benzonitrile;
2-(4-acetyl-1-piperazinyl)ethyl 7-[2-(4-cyanophenoxy)ethyl]-9-oxa-3,7-diazabicyclo[3.3.1]nonane-3-carboxylate;
7-[2-(4-cyanophenoxy)ethyl]-N-ethyl-9-oxa-3,7-diazabicyclo[3.3.1]-nonane-3-carboxamide;
4-{2-[7-(butylsulfonyl)-9-oxa-3,7-diazabicyclo[3.3.1]non-3-yl]ethoxy}benzonitrile;
4-{2-[7-(3,4-dimethoxyphenethyl)-9-oxa-3,7-diazabicyclo[3.3.1]non-3-yl]ethoxy}benzonitrile;
2-(4-acetyl-1-piperazinyl)ethyl 7-[3-(4-cyanoanilino)propyl]-9-oxa-3,7-diazabicyclo[3.3.1]nonane-3-carboxylate;
7-[3-(4-cyanoanilino)propyl]-N-ethyl-9-oxa-3,7-diazabicyclo[3.3.1]-nonane-3-carboxamide;
2-(4-acetyl-1-piperazinyl)ethyl 7-[4-(4-cyanophenyl)-4-(3,4-dimethoxyphenoxy)butyl]-9-oxa-3,7-diazabicyclo[3.3.1]nonane-3-carboxylate;
4-{3-[7-(cyclopropylmethyl)-9-oxa-3,7-diazabicyclo[3.3.1]non-3-yl]-2-hydroxypropoxy}benzonitrile;
4-(3-{7-[2-(2,3-dihydro-1,4-benzodioxin-6-yl)-2-oxoethyl]-9-oxa-3,7-diazabicyclo[3.3.1]non-3-yl}-2-hydroxypropoxy)benzonitrile;
4-(3-{7-[3-(4-acetyl-1-piperazinyl)propyl]-9-oxa-3,7-diazabicyclo[3.3.1]-non-3-yl}-2-hydroxypropoxy)benzonitrile;
2-{7-[3-(4-cyanophenoxy)-2-hydroxypropyl]-9-oxa-3,7-diazabicyclo[3.3.1]non-3-yl}-N-isopropylacetamide;
4(3-{7-[3-(ethylsulfonyl)propyl]-9-oxa-3,7-diazabicyclo[3.3.1]non-3-yl}-2-hydroxypropoxy)benzonitrile;
4-(2-hydroxy-3-{7-[2-(2-methoxyethoxy)ethyl]-9-oxa-3,7-diazabicyclo[3.3.1]non-3-yl}propoxy)benzonitrile;
4-(2-hydroxy-3-{7-[2-(4-methoxyphenyl)-2-oxoethyl]-9-oxa-3,7-diazabicyclo[3.3.1]non-3-yl}propoxy)benzonitrile;
4-({3-[7-(cyclopropylmethyl)-9-oxa-3,7-diazabicyclo[3.3.1]non-3-yl]-propyl}amino)benzonitrile;
4-[(3-{7-[2-(2,3-dihydro-1,4-benzodioxin-6-yl)-2-oxoethyl]-9-oxa-3,7-diazabicyclo[3.3.1]non-3-yl}propyl)amino]benzonitrile;
4-[(3-{7-[2-(4-methyl-1,3-thiazol-5-yl)ethyl]-9-oxa-3,7-diazabicyclo[3.3.1]non-3-yl}propyl)amino]benzonitrile;
4-[(3-{7-[3-(4-acetyl-1-piperazinyl)propyl]-9-oxa-3,7-diazabicyclo[3.3.1]-non-3-yl}propyl)amino]benzonitrile;
2-{7-[3-(4-cyanoanilino)propyl]-9-oxa-3,7-diazabicyclo[3.3.1]non-3-yl}-N-isopropylacetamide;
4-[(3-{7-[3-(ethylsulfonyl)propyl]-9-oxa-3,7-diazabicyclo[3.3.1]non-3-yl}-propyl)amino]benzonitrile;
4-[(3-{7-[2-(2-methoxyethoxy)ethyl]-9-oxa-3,7-diazabicyclo[3.3.1]non-3-yl}propyl)amino]benzonitrile;
4-({3-[7-(4-fluorobenzyl)-9-oxa-3,7-diazabicyclo[3.3.1]non-3-yl]propyl}-amino)benzonitrile;
4-[(3-{7-[2-(4-methoxyphenyl)-2-oxoethyl3-9-oxa-37-diazabicyclo3.3.1]-non-3-yl}propyl)amino]benzonitrile;
4-{2-[7-(cyclopropylmethyl)-9-oxa-3,7-diazabicyclo[3.3.1]non-3-yl]-ethoxy}benzonitrile;
4-(2-{7-[2-(2,3-dihydro-1,4-benzodioxin-6-yl)-2-oxoethyl]-9-oxa-3,7-diazabicyclo[3.3.1]non-3-yl}ethoxy)benzonitrile;
4-(2-{7-[2-(4-methyl-1,3-thiazol-5-yl)ethyl]-9-oxa-3,7-diazabicyclo[3.3.1-non-3-yl}ethoxy)benzonitrile;
4-(2-{7-[3-(4-acetyl-1-piperazinyl)propyl]-9-oxa-3,7-diazabicyclo[3.3.1]-non-3-yl}ethoxy)benzonitrile,
2-{7-[2-(4-cyanophenoxy)ethyl]-9-oxa-3,7-diazabicyclo[3.3.1]non-3-yl}-N-isopropylacetamide;
4-(2-{7-[3-(ethylsulfonyl)propyl]-9-oxa-3,7-diazabicyclo[3.3.1]non-3-yl}-ethoxy)benzonitrile;
4-(2-{7-[2-(2-methoxyethoxy)ethyl]-9-oxa-3,7-diazabicyclo[3.3.1]non-3-yl}ethoxy)benzonitrile;
4-{2-[7-(4-fluorobenzyl)-9-oxa-3,7-diazabicyclo[3.3.1]non-3-yl]ethoxy}-benzonitrile;
4-({3-[7-(3,3-dimethyl-2-oxobutyl)-9-oxa-3,7-diazabicyclo[3.3.1]non-3-yl]propyl}sulfonyl)benzonitrile;
4-({3-[7-(cyclopropylmethyl)-9-oxa-3,7-diazabicyclo[3.3.1]non-3-yl]-propyl}sulfonyl)benzonitrile;
4-[(3-{7-[2-(2,3-dihydro-1,4-benzodioxin-6-yl)-2-oxoethyl]-9-oxa-3,7-diazabicyclo[3.3.1]non-3-yl}propyl)sulfonyl]benzonitrile;
4-[(3-{7-[2-(4-methyl-1,3-thiazol-5-yl)ethyl]-9-oxa-3,7-diazabicyclo[3.3.1]non-3-yl}propyl)sulfonyl]benzonitrile;
4-[(3-{7-[3-(4-acetyl-1-piperazinyl)propyl]-9-oxa-3,7-diazabicyclo[3.3.1]-non-3-yl}propyl)sulfonyl]benzonitrile;
2-(7-{3-[(4-cyanophenyl)sulfonyl]propyl}-9-oxa-3,7-diazabicyclo[3.3.1]-non-3-yl)-N-isopropylacetamide;
4-[(3-{7-[3-(ethylsulfonyl)propyl]-9-oxa-3,7-diazabicyclo[3.3.1]non-3-yl}-propyl)sulfonyl]benzonitrile;
4-[(3-{7-[2-(2-methoxyethoxy)ethyl]-9-oxa-3,7-diazabicyclo[3.3.1]non-3-yl}propyl)sulfonyl]benzonitrile;
4-({3-[7-(4-fluorobenzyl)-9-oxa-3,7-diazabicyclo[3.3.1]non-3-yl]propyl}-sulfonyl)benzonitrile;
4-[(3-{7-(2-(4-methoxyphenyl)-2-oxoethyl]-9-oxa-3,7-diazabicyclo[3.3.1]-non-3-yl}propyl)sulfonyl]benzonitrile;
4-[(3-{7-[2-(4-fluorophenyl)-2-oxoethyl]-9-oxa-3,7-diazabicyclo[3.3.1]-non-3-yl}propyl)amino]benzonitrile;
4-(2-{7-[2-(4-fluorophenyl)-2-oxoethyl]-9-oxa-3,7-diazabicyclo[3.3.1]non-3-yl}ethoxy)benzonitrile;
4-{2-[7-(tetrahydro-2H-pyran-2-ylmethyl)-9-oxa-3,7-diazabicyclo[3.3.1-non-3-yl]ethoxy}benzonitrile;
4-(3-{7-(2-(4-fluorophenyl)-2-oxoethyl]-9-oxa-3,7-diazabicyclo[3.3.1]non-3-yl}-2-hydroxypropoxy)benzonitrile;
4-{2-hydroxy-3-[7-(tetrahydro-2H-pyran-2-ylmethyl)-9-oxa-3,7-diazabicyclo[3.3.1]non-3-yl]propoxy}benzonitrile;
4-({3-[7-(2-fluoro-3,3-dimethylbutyl)-9-oxa-3,7-diazabicyclo[3.3.1]non-3 yl]propyl}amino)benzonitrile;
4-({3-[7-(2-hydroxy-3,3-dimethylbutyl)-9-oxa-3,7-diazabicyclo[3.3.1]non-3-yl]propyl}amino)benzonitrile;
4-({3-[7-(3,3-dimethylbutyl)-9-oxa-3,7-diazabicyclo[3.3.1]non-3-yl]-propyl}amino)benzonitrile;
4-({3-[7-(2-oxopropyl)-9-oxa-3,7-diazabicyclo[3.3.1]non-3-yl]propyl}-amino)benzonitrile;
4-(2-{7-[3-(4-cyanoanilino)propyl]-9-oxa-3,7-diazabicyclo[3.3.1]non-3-yl}ethoxy)benzonitrile;
4-(2-{7-[2-(4-cyanophenoxy)ethyl]-9-oxa-3,7-diazabicyclo[3.3.1]non-3-yl}ethoxy)benzonitrile;
4-(2-{7-[2-(4-cyanophenoxy)ethyl]-9-oxa-3,7-diazabicyclo[3.3.1]non-3-yl}ethyl)benzonitrile;
4-{4-[7-(3,3-dimethyl-2-oxobutyl)-9-oxa-3,7-diazabicyclo[3.3.1]non-3-yl]-butyl}benzonitrile;
4-{2-[7-(2-phenoxyethyl)-9-oxa-3,7-diazabicyclo[3.3.1]non-3-yl]ethoxy}-benzonitrile;
2-{7-[2-(4-cyanophenoxy)ethyl]-9-oxa-3,7-diazabicyclo[3.3.1]non-3-yl}-N,N-diethylacetamide;
4-[(3-{7-[4-(4-fluorophenyl)-4-oxobutyl]-9-oxa-3,7-diazabicyclo[3.3.1]-non-3-yl}propyl)amino]benzonitrile;
4-({7-[3-(4-cyanoanilino)propyl]-9-oxa-3,7-diazabicyclo[3.3.1]non-3-yl}-methyl)benzonitrile;
4-{2-[7-(2,4-difluorobenzyl)-9-oxa-3,7-diazabicyclo[3.3.1]non-3-yl]-ethoxy}benzonitrile;
4-[(3-{7-[4-(difluoromethoxy)benzyl]-9-oxa-3,7-diazabicyclo[3.3.1]non-3-yl}propyl)amino]benzonitrile;
4-[(3-{7-[2-(1H-pyrrol-1-yl)ethyl]-9-oxa-3,7-diazabicyclo[3.3.1]non-3-yl}-propyl)amino]benzonitrile;
4-[(3-{7-[3-(4-bromophenyl)-3-oxopropyl]-9-oxa-3,7-diazabicyclo[3.3.1]-non-3-yl}propyl)amino]benzonitrile;
4-{2-[7-(2,2-difluoroethyl)-9-oxa-3,7-diazabicyclo[3.3.1]non-3-yl]ethoxy}-benzonitrile;
4-({3-[7-(2-phenoxyethyl)-9-oxa-3,7-diazabicyclo[3.3.1]non-3-yl]propyl}-amino)benzonitrile;
4-(2-{7-[2-(1H-pyrrol-1-yl)ethyl]-9-oxa-3,7-diazabicyclo[3.3.1]non-3-yl}-ethoxy)benzonitrile;
4-[((2S)-3-{7-[(2S)-3-(4-cyanophenoxy)-2-hydroxypropyl]-9-oxa-3,7-diazabicyclo[3.3.1]non-3-yl}-2-hydroxypropyl)oxy]benzonitrile;
4-[((2S)-2-hydroxy-3-{7-[2-(1H-pyrrol-1-yl)ethyl]-9-oxa-3,7-diazabicyclo[3.3.1]non-3-yl}propyl)oxy]benzonitrile;
4-{2-[7-(3,3-dimethyl-2-oxobutyl)-9-oxa-3,7-diazabicyclo[3.3.1]non-3-yl]-ethoxy}isophthalonitrile;
4-(2-{7-[2-(4-methoxyphenyl)-2-oxoethyl]-9-oxa-3,7-diazabicyclo[3.3.1]-non-3-yl}ethoxy)isophthalonitrile;
4-(2-{7-[2-(1H-pyrrol-1-yl)ethyl]-9-oxa-3,7-diazabicyclo[3.3.1]non-3-yl}-ethoxy)isophthalonitrile;
tert-butyl 2-{7-[2-(2,4-dicyanophenoxy)ethyl]-9-oxa-3,7-diazabicyclo[3.3.1]non-3-yl}ethylcarbamate;
4-({(2S)-2-amino-3-[7-(3,3-dimethyl-2-oxobutyl)-9-oxa-3,7-diazabicyclo-[3.3.1]non-3-yl]propyl}oxy)benzonitrile;
4-[((2S)-2-amino-3-{7-[2-(4-methoxyphenyl)-2-oxoethyl]-9-oxa-3,7-diazabicyclo[3.3.1]non-3-yl}propyl)oxy]benzonitrile;
4-{3-[7-(3,3-dimethyl-2-oxobutyl)-9-oxa-3,7-diazabicyclo[3.3.1]non-3-yl]-propoxy}benzonitrile;
4-(3-{7-[2-(4-fluorophenyl)-2-oxoethyl]-9-oxa-3,7-diazabicyclo[3.3.1]non-3-yl}propoxy)benzonitrile;
4-(3-{7-[2-(1H-pyrrol-1-yl)ethyl]-9-oxa-3,7-diazabicyclo[3.3.1]non-3-yl}-propoxy)benzonitrile;
4-(4-{7-[2-(1H-pyrrol-1-yl)ethyl]-9-oxa-3,7-diazabicyclo[3.3.1]non-3-yl}-butyl)benzonitrile;
4-{[(2S)-3-(7-{2-[4-(tert-butoxy)phenoxy]ethyl}-9-oxa-3,7-diazabicyclo[3.3.1]non-3-yl)-2-hydroxypropyl]oxy}benzonitrile;
4-[((2S)-3-{7-[2-(3,5-dimethyl-1H-pyrazol-1-yl)ethyl]-9-oxa-3,7-diazabicyclo[3.3.1]non-3-yl}-2-hydroxypropyl)oxy]benzonitrile;
4-{3-[7-(imidazo[1,2-a]pyridin-2-ylmethyl)-9-oxa-3,7-diazabicyclo[3.3.1]-non-3-yl]propoxy}benzonitrile;
4-{3-[7-(2-phenoxyethyl)-9-oxa-3,7-diazabicyclo[3.3.1]non-3-yl]propoxy}-benzonitrile;
4-(3-{7-[2-(3,5-dimethyl-1H-pyrazol-1-yl)ethyl]-9-oxa-3,7-diazabicyclo[3.3.1]non-3-yl}propoxy)benzonitrile;
4-({3-[7-(imidazo[1,2-a]pyridin-2-ylmethyl)-9-oxa-3,7-diazabicyclo[3.3.1]non-3-yl]propyl}amino)benzonitrile;
4-({3-[7-(2,4-difluorobenzyl)-9-oxa-3,7-diazabicyclo[3.3.1]non-3-yl]-propyl}amino)benzonitrile;
4-{[3-(7-{2-[4-(tert-butoxy)phenoxy]ethyl}-9-oxa-3,7-diazabicyclo[3.3.1]-non-3-yl)propyl]amino}benzonitrile;
4-{2-[7-(imidazo[1,2-a]pyridin-2-ylmethyl)-9-oxa-3,7-diazabicyclo[3.3.1]-non-3-yl]ethoxy}benzonitrile;
tert-butyl 2-{7-[2-(4-cyanophenoxy)ethyl]-9-oxa-3,7-diazabicyclo[3.3.1]-non-3-yl}ethylcarbamate;
4-{[3-(7-{2-[4-(tert-butoxy)phenoxy]ethyl}-9-oxa-3,7-diazabicyclo[3.3.1]-non-3-yl)propyl]sulfonyl}benzonitrile;
4-[(3-{7-[2-(3,5-dimethyl-1H-pyrazol-1-yl)ethyl]-9-oxa-3,7-diazabicyclo[3.3.1]non-3-yl}propyl)sulfonyl]benzonitrile;
4-({3-[7-(2,4-difluorobenzyl)-9-oxa-3,7-diazabicyclo[3.3.1]non-3-yl]-propyl}sulfonyl)benzonitrile;
4-{2-[7-(imidazo[1,2-a]pyridin-2-ylmethyl)-9-oxa-3,7-diazabicyclo[3.3.1]-non-3-yl]ethoxy}isophthalonitrile;
4-[2-(7-{2-[4-(tert-butoxy)phenoxy]ethyl}-9-oxa-3,7-diazabicyclo[3.3.1]-non-3-yl)ethoxy]isophthalonitrile;
4-(2-{7-[2-(3,5-dimethyl-1H-pyrazol-1-yl)ethyl]-9-oxa-3,7-diazabicyclo[3.3.1]non-3-yl}ethoxy)isophthalonitrile;
4-(4-{7-[2-(1H-imidazol-4-yl)ethyl]-9-oxa-3,7-diazabicyclo[3.3.1]non-3-yl}butyl)benzonitrile;
4-{4-[7-(imidazo[1,2-a]pyridin-2-ylmethyl)-9-oxa-3,7-diazabicyclo[3.3.1]-non-3-yl]butyl}benzonitrile;
4-{4-[7-(2-phenoxyethyl)-9-oxa-3,7-diazabicyclo[3.3.1]non-3-yl]butyl}-benzonitrile;
4-(4-{7-[2-(3,5-dimethyl-1H-pyrazol-1-yl)ethyl]-9-oxa-3,7-diazabicyclo[3.3.1]non-3-yl}butyl)benzonitrile;
4-[3-(7-{2-oxo-2-[4-(1-pyrrolidinyl)phenyl]ethyl}-9-oxa-3,7-diazabicyclo-[3.3.1]non-3-yl)propoxy]benzonitrile;
4-(3-{7-[2-(4-hydroxyphenyl)-2-oxoethyl]-9-oxa-3,7-diazabicyclo[3.3.1]-non-3-yl}propoxy)benzonitrile;
4-(3-{7-[2-(4-methylphenyl)-2-oxoethyl]-9-oxa-3,7-diazabicyclo[-3.3.1-]-non-3-yl}propoxy)benzonitrile;
4-(3-{7-[2-(4-methoxyphenyl)-2-oxoethyl]-9-oxa-3,7-diazabicyclo[3.3.1]-non-3-yl}propoxy)benzonitrile;
4-(3-{7-[2-(2,3-dihydro-1,4-benzodioxin-6-yl)-2-oxoethyl]-9-oxa-3,7-diazabicyclo[3.3.1]non-3-yl}propoxy)benzonitrile;
4-(2-{7-[2-(2,6-dimethylphenoxy)-1-methylethyl]-9-oxa-3,7-diazabicyclo-[3.3.1]non-3-yl}ethoxy)benzonitrile;
4-(3-{7-[2-oxo-2-(3-oxo-3,4-dihydro-2H-1,4-benzoxazin-6-yl)ethyl]-9-oxa-3,7-diazabicyclo[3.3.1]non-3-yl}propoxy)benzonitrile;
tert-butyl 2-{7-[3-(4-cyanophenoxy)propyl]-9-oxa-3,7-diazabicyclo[3.3.1]-non-3-yl}ethylcarbamate;
N-(tert-butyl)-N′-(2-{7-[3-(4-cyanophenoxy)propyl]-9-oxa-3,7-diazabi-cyclo[3.3.1]non-3-yl}ethyl)urea;
tert-butyl 2-({7-[2-(4-cyanophenoxy)ethyl]-9-oxa-3,7-diazabicyclo[3.3.1]-non-3-yl}methyl)-1-pyrrolidinecarboxylate;
4-{[3-(7-benzyl-9-oxa-3,7-diazabicyclo[3.3.1]non-3-yl)propyl]amino}-benzonitrile;
4-[(3-{7-[3-(4-cyanoanilino)propyl]-9-oxa-3,7-diazabicyclo[3.3.1]non-3-yl}propyl)amino]benzonitrile;
tert-butyl 2-{7-[2-(4-nitrophenoxy)ethyl]-9-oxa-3,7-diazabicyclo[3.3.1]-non-3-yl}ethylcarbamate (m/z=437);
tert-butyl 2-[7-(2-{4-[(methylsulfonyl)amino]phenoxy}ethyl)-9-oxa-3,7-diazabicyclo[3.3.1]non-3-yl]ethylcarbamate;
tert-butyl 2-{7-[2-(4-aminophenoxy)ethyl]-9-oxa-3,7-diazabicyclo[3.3.1]-non-3-yl}ethylcarbamate;
4-({3-[7-(phenylsulfonyl)-9-oxa-3,7-diazabicyclo[3.3.1]non-3-yl]propyl}-amino)benzonitrile; or
4-({3-[7-(3,3-dimethyl-2-oxobutyl)-9-oxa-3,7-diazabicyclo[3.3.1]non-3-yl]propyl}amino)benzamide.

This list of compounds and including pharmaceutically acceptable derivatives of the compounds as defined in WO 01/28992 will hereinafter be referred to as a compound of Claim 34 of WO 01/28992.

PCT/SE02/00724 discloses modified release formulations of the following compounds which are described in WO 01/28992:

(a) 4-({3-[7-(3,3-dimethyl-2-oxobutyl)-9-oxa-3,7-diazabicyclo[3.3.1]non-3-yl]propyl}amino)benzonitrile: 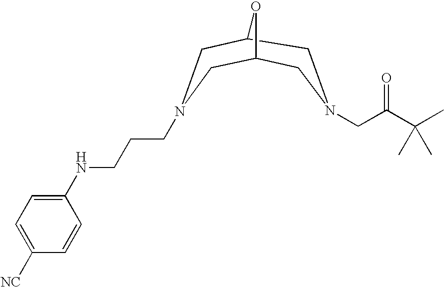
which compound is referred to hereinafter as Compound A. Compound A is specifically disclosed in WO 01/28992 both in the form of the free base and in the form of a benzenesulphonate salt;

(b) tert-butyl 2-{7-[3-(4-cyanoanilino)propyl]-9-oxa-3,7-diazabicyclo-[3.3.1]non-3-yl}ethylcarbamate: 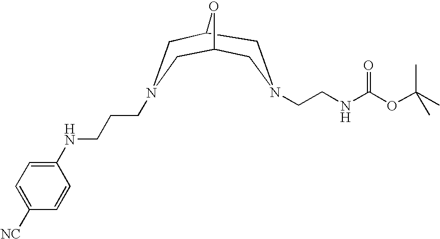
in the form of the free base, which compound is referred to hereinafter as Compound B;

(c) tert-butyl 2-{7-[4-(4-cyanophenyl)butyl]-9-oxa-3,7-diazabicyclo-[3.3.1]non-3-yl}ethylcarbamate: 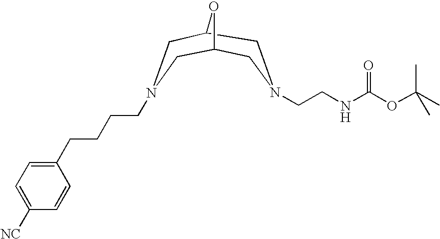
in the form of the free base, which compound is referred to hereinafter as Compound C; and

(d) tert-butyl 2-{7-[(2S)-3-(4-cyanophenoxy)-2-hydroxypropyl]-9-oxa-3,7-diazabicyclo[3.3.1]non-3-yl}ethylcarbamate: 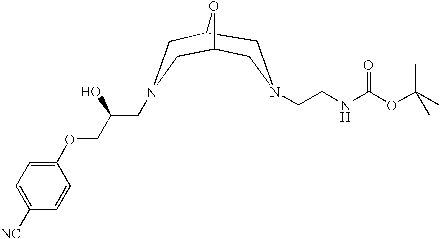
in the form of the free base, which compound is referred to hereinafter as Compound D.

Current drug therapies for AF include antiarryhthmic drugs, administered with a view to re-establishing and maintaining a normal heartbeat or to controlling heart rate, and anticoagulant and/or thrombolytic drugs, administered with a view to preventing thromboembolism and/or cerebral stroke.

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V and factor VIII leading to a “positive feedback” generation of thrombin from prothrombin.

International patent application WO 94/29336 discloses a group of thrombin-inhibiting compounds, including HOOC—CH₂—(R)Cgl-Aze-Pab-H (in which Cgl represents cyclohexylglycine, Aze represents S-azetidine-2-carboxylic acid and Pab-H represents 4-aminomethylamidinobenzene), which is also known as melagatran (see Example 1 of WO 94/29336). International Patent Application WO 97/23499 discloses prodrugs of inter alia melagatran.

However, it is estimated that only 40% of patients with AF who should benefit from anticoagulant therapy do so, owing to the risks associated with existing treatments. This also includes patients whose anticoagulant therapy is in combination with cardioversion (electrical or chemical). In particular, of the currently-available oral anticoagulants, warfarin (a vitamin K antagonist) carries the risk of bleeding, and the need for frequent laboratory control. Vitamin K antagonists also demonstrate a notable risk of interaction with other drugs and certain foods, e.g. those that are rich in Vitamin K, and their use requires monitoring of the patient's blood coagulation status. Medication containing acetylsalicylic acid (an antiplatelet agent) also carries the risk of bleeding. Blood coagulation is the key process involved in both haemostasis (i.e. the prevention of blood loss from a damaged vessel) and thrombosis (i.e. the formation of a blood clot in a blood vessel, sometimes leading to vessel obstruction).

There remains a need for a combination of an antiarrhythmic drug and an anti-coagulant drug that has fewer side-effects than existing therapies and will encourage the use of such a combination in a higher percentage of AF patients.

None of the above-mentioned documents disclose or suggest the administration of melagatran or a pharmaceutically-acceptable derivative thereof in conjunction with a compound as defined in claim 1 of WO 01/28992. Surprisingly, the administration of just such a combination gives rise to unexpected, beneficial effects.

### DISCLOSURE OF THE INVENTION

According to a first aspect of the invention there is provided a combination product comprising:

(1) melagatran or a pharmaceutically-acceptable derivative thereof; and

(2) a compound as defined in claim 1 of WO 01/28992.

According to a second aspect of the invention there is provided a combination product comprising:

(1) melagatran or a pharmaceutically-acceptable derivative thereof; and

(2) a compound of Claim 34 of WO 01/28992.

According to a third aspect of the invention there is provided a combination product comprising:

(1) melagatran or a pharmaceutically-acceptable derivative thereof; and

(2) (a) 4-({3-[7-(3,3-dimethyl-2-oxobutyl)-9-oxa-3,7-diazabicyclo[3.3.1]non-3-yl]propyl}amino)benzonitrile: 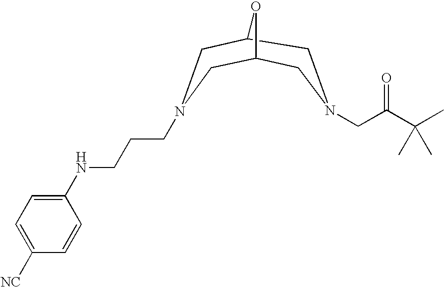
which compound is referred to hereinafter as Compound A or a pharmaceutically-acceptable salt thereof; or

(b) tert-butyl 2-{7-[3-(4-cyanoanilino)propyl]-9-oxa-3,7-diazabicyclo-[3.3.1]non-3-yl}ethylcarbamate: 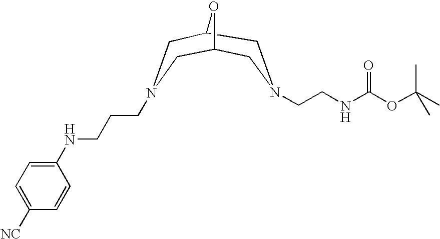
in the form of the free base, which compound is referred to hereinafter as Compound B or a pharmaceutically-acceptable salt thereof; or

(c) tert-butyl 2-{7-[4-(4-cyanophenyl)butyl]-9-oxa-3,7-diazabicyclo-[3.3.1]non-3-yl}ethylcarbamate: 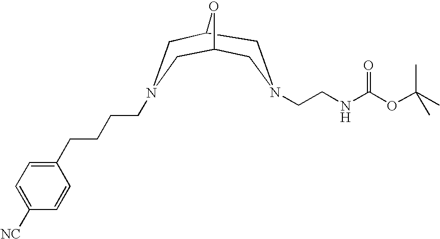
in the form of the free base, which compound is referred to hereinafter as Compound C or a pharmaceutically-acceptable salt thereof; or

(d) tert-butyl 2-{7-[(2S)-3-(4-cyanophenoxy)-2-hydroxypropyl]-9-oxa-3,7-diazabicyclo[3.3.1]non-3-yl}ethylcarbamate: 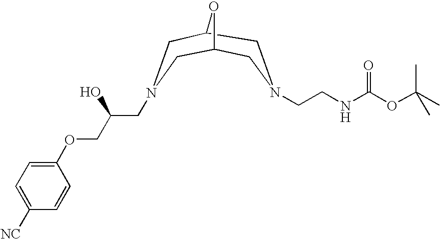
in the form of the free base, which compound is referred to hereinafter as Compound D or a pharmaceutically-acceptable salt thereof;
wherein each of components (1) and (2) is formulated in admixture with a pharmaceutically-acceptable adjuvant, diluent or carrier.

The combination product according to the invention provides for the administration of melagatran (or derivative thereof) in conjunction with (1) a compound as defined in claim 1 of WO 01/28992 or (2) a compound of Claim 34 of WO 01/28992 or (3) Compound A or B or C or D (or pharmaceutically-acceptable salts thereof), and may thus be presented either as separate formulations, wherein at least one of those formulations comprises melagatran and at least one comprises (1) a compound as defined in claim 1 of WO 01/28992 or (2) a compound of Claim 34 of WO 01/28992 or Compound A or B or C or D (or pharmaceutically-acceptable salts thereof), or may be presented (i.e. formulated) as a combined preparation (i.e. presented as a single formulation including melagatran and (1) a compound as defined in claim 1 of WO 01/28992 or (2) a compound of Claim 34 of WO 01/28992 or (3) Compound A or B or C or D (or pharmaceutically-acceptable salts thereof)).

Thus, there is further provided:

(1) a pharmaceutical formulation including melagatran or a pharmaceutically-acceptable derivative thereof, and (1) a compound as defined in claim 1 of WO 01/28992 or (2) a compound of Claim 34 of WO 01/28992 or (3) Compound A or B or C or D (or pharmaceutically-acceptable salts thereof), in admixture with a pharmaceutically-acceptable adjuvant, diluent or carrier (which formulation is hereinafter referred to as a “combined preparation”); and

(2) a kit of parts comprising components:

(a) a pharmaceutical formulation including melagatran or a pharmaceutically-acceptable derivative thereof, in admixture with a pharmaceutically-acceptable adjuvant, diluent or carrier; and

(b) a pharmaceutical formulation including (1) a compound as defined in claim 1 of WO 01/28992 or (2) a compound of Claim 34 of WO 01/28992 or (3) Compound A or B or C or D (or pharmaceutically-acceptable salts thereof) in admixture with a pharmaceutically-acceptable adjuvant, diluent or carrier,

which components (a) and (b) are each provided in a form that is suitable for administration in conjunction with the other.

According to a further aspect of the invention, there is provided a method of making a kit of parts as defined above, which method comprises bringing a component (a), as defined above, into association with a component (b), as defined above, thus rendering the two components suitable for administration in conjunction with each other.

By bringing the two components “into association with” each other, we include that components (a) and (b) of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Thus, there is further provided a kit of parts comprising:

(I) one of components (a) and (b) as defined herein; together with

(II) instructions to use that component in conjunction with the other of the two components.

The kits of parts described herein may comprise more than one formulation including an appropriate quantity/dose of melagatran or derivative thereof, and/or more than one-formulation including an appropriate quantity/dose of (1) a compound as defined in claim 1 of WO 01/28992 or (2) a compound of Claim 34 of WO 01/28992 or (3) Compound A or B or C or D (or pharmaceutically-acceptable salts thereof), in order to provide for repeat dosing. If more than one formulation (comprising either active compound) is present, such formulations may be the same, or may be different in terms of the dose of melagatran (or derivative) or (1) a compound as defined in claim 1 of WO 01/28992 or (2) a compound of Claim 34 of WO 01/28992 or (3) Compound A or B or C or D (or pharmaceutically-acceptable salts thereof), chemical composition and/or physical form.

A further aspect of the invention provides a method of treatment of a condition where anticoagulant therapy is indicated, which comprises administration of a pharmaceutical formulation including melagatran (or a pharmaceutically-acceptable derivative thereof), and (1) a compound as defined in claim 1 of WO 01/28992 or (2) a compound of Claim 34 of WO 01/28992 or (3) Compound A or B or C or D (or pharmaceutically-acceptable salts thereof), in admixture with a pharmaceutically-acceptable adjuvant, diluent or carrier.

A further aspect of the invention provides a method of treatment of a condition where anticoagulant therapy is indicated (by which we mean where anticoagulation is required), which comprises administration of:

(a) a pharmaceutical formulation including melagatran or a pharmaceutic ally-acceptable derivative thereof, in admixture with a pharmaceutically-acceptable adjuvant, diluent or carrier; in conjunction with

(b) a pharmaceutical formulation including (1) a compound as defined in claim 1 of WO 01/28992 or (2) a compound of Claim 34 of WO 01/28992 or (3) Compound A or B or C or D (or pharmaceutically-acceptable salts thereof), in admixture with a pharmaceutically-acceptable adjuvant, diluent or carrier,

to a patient suffering from, or susceptible to, such a condition.

For the avoidance of doubt, as used herein, the term “treatment” includes therapeutic and/or prophylactic treatment.

With respect to the kits of parts as described herein, by “administration in conjunction with”, we include that respective formulations comprising melagatran (or derivative thereof) and (1) a compound as defined in claim 1 of WO 01/28992 or (2) a compound of Claim 34 of WO 01/28992 or (3) Compound A or B or C or D (or pharmaceutically-acceptable salts thereof), are administered, sequentially, separately and/or simultaneously, over the course of treatment of the relevant condition, which condition may be acute or chronic.

Thus, in respect of the combination product according to the invention, the term “administration in conjunction with” includes that the two components of the combination product (melagatran/derivative and (1) a compound as defined in claim 1 of WO 01/28992 or (2) a compound of Claim 34 of WO 01/28992 or (3) Compound A or B or C or D (or pharmaceutically-acceptable salts thereof)) are administered (optionally repeatedly), either (in the case of a combined preparation) together, or (in the case of a kit of parts) sufficiently closely in time, to enable a beneficial effect for the patient, that is greater, over the course of the treatment of the relevant condition, than if either a formulation comprising melagatran/derivative, or a formulation comprising (1) a compound as defined in claim 1 of WO 01/28992 or (2) a compound of Claim 34 of WO 01/28992 or (3) Compound A or B or C or D (or pharmaceutically-acceptable salts thereof), are administered (optionally repeatedly) alone, in the absence of the other component, over the same course of treatment. Determination of whether a combination provides a greater beneficial effect in respect of, and over the course of treatment of, a particular condition, will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration with the other component. When used in this context, the terms “administered simultaneously” and “administered at the same time as” include that individual doses of melagatran (or derivative thereof) and (1) a compound as defined in claim 1 of WO 01/28992 or (2) a compound of Claim 34 of WO 01/28992 or (3) Compound A or B or C or D (or pharmaceutically-acceptable salts thereof), are administered within 48 hours (e.g. 24 hours) of each other.

“Pharmaceutically-acceptable derivatives” of melagatran includes salts (e.g. pharmaceutically-acceptable non-toxic organic or inorganic acid addition salts) and solvates. It will be appreciated that the term further includes derivatives that have the same biological function and/or activity as melagatran, as appropriate. Moreover, for the purposes of this invention, the term also includes prodrugs of melagatran. “Prodrugs” of melagatran include any composition of matter that, following oral or parenteral administration, is metabolised in vivo to form either melagatran, as appropriate, in an experimentally-detectable amount, and within a predetermined time (e.g. within a dosing interval of between 6 and 24 hours (i.e. once to four times daily)). For the avoidance of doubt, the term “parenteral” adminstration includes all forms of adminstration other than oral administration.

Prodrugs of melagatran that may be mentioned include those disclosed in international patent application WO 97/23499. Preferred prodrugs are those of the formula R¹O₂C—CH₂—(R)Cgl-Aze-Pab-OH (see the list of abbreviations above or in WO 97/23499), wherein R¹ represents C₁₋₁₀ alkyl or benzyl, such as linear or branched C₁₋₆ alkyl (e.g. C₁₋₄ alkyl, especially methyl, n-propyl, i-propyl, t-butyl and, particularly, ethyl) and the OH group replaces one of the amidino hydrogens in Pab. A particularly preferred prodrug is EtO₂C—CH₂—RCgl-Aze-Pab-OH; Example 17 of WO 97/23499; Glycine, N-[1-cyclohexyl-2-[2-[[[[4-[(hydroxyimino)aminomethyl]-phenyl]methyl]amino]carbonyl]-1-azetidinyl]-2-oxoethyl]-, ethyl ester, [S-(R*, S*)]-.

Suitable doses of melagatran and pharmaceutically-acceptable derivatives thereof, (1) a compound as defined in claim 1 of WO 01/28992 or (2) a compound of Claim 34 of WO 01/28992 or (3) Compound A or B or C or D (or pharmaceutically-acceptable salts thereof), in the therapeutic and/or prophylactic treatment of mammalian, especially human, patients may be determined routinely by the medical practitioner or other skilled person, and include the respective doses discussed in the prior art documents relating to melagatran (or derivatives (including prodrugs) thereof), and antiarrhythmic oxabispidines, that are mentioned hereinbefore, the disclosures in which documents are hereby incorporated by reference.

In the case of melagatran, suitable doses of active compound, prodrugs and derivatives thereof, in the therapeutic and/or prophylactic treatment of mammalian, especially human, patients include those which give a mean plasma concentration of up to 5 Tmol/L, for example in the range 0.001 to 5 Tmol/L over the course of treatment of the relevant condition. Suitable doses may thus be in the range 0.1 mg once daily to 25 mg three times daily, and/or up to 100 mg infused parenterally over a 24 hour period, for melagatran, and in the range 0.1 mg once daily to 100 mg three times daily for prodrugs of melagatran including those specifically mentioned hereinbefore. In the case where the prodrug is EtO₂C—CH₂—RCgl-Aze-Pab-OH then the preferred dose is selected from 12 mg, 24 mg, 36 mg, 48 mg, 60 mg or 72 mg.

In the case of antiarrhythmic oxabispidines typical daily doses of (1) a compound as defined in claim 1 of WO 01/28992 or (2) a compound of Claim 34 of WO 01/28992 or (3) Compound A or B or C or D (or pharmaceutically-acceptable salts thereof), are in the range 10 to 2000 mg, e.g. 25, such as 30, to 1200 mg of free base (i.e., in the case of a salt, excluding any weight resulting from the presence of a counter ion), irrespective of the number of compositions (e.g. tablets) that are administered during the course of that day. Preferred daily doses are in the range 50 to 1000 mg, such as 100 to 500 mg, for example 150 mg, 200 mg, 250 mg, 300 mg, 350 mg,400 mg or 450 mg. Typical doses in individual compositions of the invention (e.g. tablets) are thus in the range 15 to 500 mg, for example 40 to 400 mg eg for example 150 mg, 200 mg, 250 mg, 300 mg, 350 mg or 400 mg.

Specifically claimed herein are specific fixed dose combinations where any dose stated for melagatran and is combined with any dose stated for the antiarrhythmic oxabispidine, including the doses stated as limits for the ranges described.

In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

When separate formulations are administered, the sequence in which the formulations comprising melagatran (or derivative thereof), and the antiarrhythmic oxabispidine (or derivative thereof), may be administered (i.e. whether, and at what point, sequential, separate and/or simultaneous administration takes place) may be determined by the physician or skilled person. For example, the sequence may depend upon many factors that will be evident to the skilled person, such as whether, at any time during the course or period of treatment, one or other of the formulations cannot be administered to the patient for practical reasons (e.g. the patient is unconscious and thus unable to take an oral formulation comprising either melagatran or the antiarrhythmic oxabispidine).

The method described herein may have the advantage that, in the treatment of conditions where anticoagulant therapy is indicated, it may be more convenient for the physician and/or patient than, be more efficacious than, be less toxic than, have a broader range of activity than, be more potent than, produce fewer side effects than, or that it may have other useful pharmacological properties over, similar methods known in the prior art for the treatment of such conditions.

Melagatran, and derivatives thereof, may be administered for systemic delivery using appropriate means of administration that are known to the skilled person.

Thus, in accordance with the invention, melagatran, and derivatives thereof, may be administered orally, intravenously, subcutaneously, buccally, rectally, dermally, nasally, tracheally, bronchially, topically, by any other parenteral route, or via inhalation, in the form of a pharmaceutical preparation comprising the active ingredient in a pharmaceutically-acceptable dosage form. Depending on the disorder, and the patient, to be treated, as well as the route of administration, the compositions may be administered at varying doses.

Preferred modes of delivery are systemic. For melagatran, preferred modes of administration are parenteral, more preferably intravenous, and especially subcutaneous. For prodrugs of melagatran, preferred modes of administration are oral.

In the therapeutic treatment of mammals, and especially humans, melagatran and derivatives thereof may be administered alone, but will generally be administered as a pharmaceutical formulation in admixture with a pharmaceutically-acceptable adjuvant, diluent or carrier, which may be selected with due regard to the intended route of administration and standard pharmaceutical practice.

Suitable formulations for use in administering melagatran and derivatives (including prodrugs) thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference. Otherwise, the preparation of suitable formulations may be achieved non-inventively by the skilled person using routine techniques.

The combinations of the present invention are useful in both the prophylaxis and the treatment of cardiac arrhythmias, in particular atrial and ventricular arrhythmias (such as atrial fibrillation (e.g. atrial flutter)) and NVAF.

The combinations of the invention are thus indicated in the treatment or prophylaxis of cardiac diseases, or in indications related to cardiac diseases, in which arrhythmias are believed to play a major role, including ischemic heart disorders, sudden heart attack, myocardial infarction, heart failure, cardiac surgery and thromboembolic events.

The term “ischemic disorders” will be understood by those skilled in the art to include any condition, the results of which include a restriction in blood flow in a part of the body. In this context, the term will also be understood to include thrombosis and hypercoagulability in blood and/or organs, tissues, etc.

The term “thrombosis” will be understood by those skilled in the art to include the formation, development or presence of a thrombus in animals including man, and which may result in embolism and/or ischemia. The term may thus include conditions such as atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

The term “hypercoagulability” includes any state in which the blood is more readily coagulated than usual.

The term “NVAF” may be understood by those skilled in the art to mean grossly disorganised atrial electrical activity, which is irregular in respect of both rate and rhythm, leading to a hypercoagulable state and an increased risk of thrombosis originating from the left heart chambers, and particularly the left atrium. The term may thus also be understood to include AF (chronic, persistent, permanent and/or intermittent (paroxysmal)) in the absence of heart valvular disease (mostly rheumatic heart valvular disease e.g. mitral stenosis), or prosthesis, and to exclude patients with rheumatic mitral stenosis.

Particular disease states that may be mentioned include the prevention/treatment of ischemic heart disease, myocardial infarction, systemic embolic events in e.g. the kidneys, spleen etc, and, more particularly, of cerebral ischemia, including cerebral thrombosis, cerebral embolism and/or cerebral ischemia associated with non-cerebral thrombosis or embolism (in other words, the treatment/prophylaxis of thrombotic, or ischemic, stroke and of transient ischemic attack (TIA)) in patients with, or at risk of, NVAF. The skilled person will appreciate that patients with NVAF who are at risk of stroke include elderly patients generally (e.g. those with an age of greater than 75 years); patients with complicating health factors, such as hypertension, left ventricular dysfunction (e.g. left ventricular ejection fraction (LVEF) of less than 40%), symptomatic congestive heart failure, diabetes mellitus (especially in those patients of 65 years of age or greater) and/or coronary heart or artery disease (especially in those patients of 65 years of age or greater); and/or patients with a history of stroke, TIA and/or systemic embolism, all of which factors may predispose such patients to stroke and/or thromboembolic events.

According to a further aspect of the invention, there is provided a method of treatment of an arrhythmia which method comprises administration of a combination of the invention to a person suffering from, or susceptible to, such a condition.

According to a further aspect of the invention, there is provided a method of treatment of atrial fibrillation which method comprises administration of a combination of the invention to a person suffering from, or susceptible to, such a condition.

According to a further aspect of the invention, there is provided a method of treatment of atrial flutter which method comprises administration of a combination of the invention to a person suffering from, or susceptible to, such a condition.

For the avoidance of doubt, by “treatment” we include the therapeutic treatment, as well as the prophylaxis, of a condition.

It is expected that the combinations of the present invention may provide one or more of the following advantages. Synergy between the components in terms of:

response rate

patient survival rate

time to disease progression

dose/response effects leading to lower doses with same efficacy.

Alternatively, it is expected that the combinations of the present invention may provide one or more of the following advantages:

lower toxicity/reduced side effects with similar/improved efficacy;

improved physical properties, e.g. storage stability, flow properties etc.;

ease of formulation for example, reduced drug/drug incompatibility problems;

reduced drug/drug interaction problems on administration, for example possible changes in metabolism of one drug caused by the effect of the other drug;

improved patient compliance;

improved quality of life;

covenient dosing regimes; or

lack of diminishing effects of one drug caused by the presence of the other drug.

It is expected that the combination of the present invention will lead to a reduced incidence of strokes in patients suspectible to strokes by the treatment and prevention of atrial fibrillation.

Improved patient compliance may be demonstrated by methods known to those skilled in the art, for example by supplying patients with blister packs containing the combination of the present invention wherein the date and time of the removal of a drug from the blister pack is recorded.

In a further aspect the present invention provides a process for the preparation of a combination product as described earlier comprising formulating (1) a dose of melagatran or a pharmaceutically-acceptable derivative thereof as previously described herein with a pharmaceutically acceptable diluent or carrier; and then formulating (1) a compound as defined in claim 1 of WO 01/28992 or (2) a compound of Claim 34 of WO 01/28992 or (3) Compound A or B or C or D (or pharmaceutically-acceptable salts thereof) in a dose as previously described herein with a pharmaceutically acceptable diluent or carrier; and then combining these formulations to provide a combination product as previously described herein.

The combination product of the present invention can be used both in conversion of AF into normal sinus rhytm and maintenance of said sinus rhytm.

The combination product of the present invention can be used to treat both symptomatic and asymptomatic atrial fibrillation.

The combination product of the present invention can be used to treat paroxysmal AF, persistent AF and permanent AF.

The ratios of the active compound in the combination product of the present invention can be in the range of 100:1, 50:1, 20:1, 10:1, 5:1, 2:1, 1:1, 1:2, 1:5, 1:10, 1:50 or 1:100.

The present invention therefore provides the additional advantage that it allows tailoring of treatment to the needs of a particular patient population. Examples of such particular patient population are; 1) elderly patient, especially over the age of 60, preferably over the age of 70, more preferably over the age of 80; 2) female patients; 3) patients suffering from any of the following conditions; hypertension, heart failure, and diabetes.

The combination product of the present invention, is either additive or synergistic in effect in the treatment of AF, in particular paroxysmal AF, persistent AF and permanent AF of a particular patient population. Examples of such particular patient population are; 1) elderly patient, especially over the age of 60, preferably over the age of 70, more preferably over the age of 80; 2) female patients; 3) patients suffering from any of the following conditions; hypertension, heart failure, and diabetes.

1. A combination product comprising:
(a) melagatran or a pharmaceutically-acceptable derivative thereof; and
(b) (1) a compound as defined in claim 1 of WO 01/28992 or (2) a compound of claim 34 of WO 01/28992 or (3) Compound A or B or C or D (or pharmaceutically-acceptable salts thereof)
wherein each of components (a) and (b) is formulated in admixture with a pharmaceutically-acceptable adjuvant, diluent or carrier.

2. A combination product as claimed in claim 1 which comprises a pharmaceutical formulation including melagatran or a pharmaceutically-acceptable derivative thereof, and (1) a compound as defined in claim 1 of WO 01/28992 or (2) a compound of claim 34 of WO 01/28992 or (3) Compound A or B or C or D (or pharmaceutically-acceptable salts thereof) or a pharmaceutically-acceptable derivative thereof, in admixture with a pharmaceutically-acceptable adjuvant, diluent or carrier.

3. A combination product as claimed in claim 1 which comprises a kit of parts comprising components:
(a) a pharmaceutical formulation including melagatran or a pharmaceutically-acceptable derivative thereof, in admixture with a pharmaceutically-acceptable adjuvant, diluent or carrier; and
(b) a pharmaceutical formulation including (1) a compound as defined in claim 1 of WO 01/28992 or (2) a compound of claim 34 of WO 01/28992 or (3) Compound A or B or C or D (or pharmaceutically-acceptable salts thereof), in admixture with a pharmaceutically-acceptable adjuvant, diluent or carrier, which components (a) and (b) are each provided in a form that is suitable for administration in conjunction with the other.

4. A kit of parts as claimed in claim 3, wherein components (a) and (b) are suitable for sequential, separate and/or simultaneous use in the treatment of a condition where anticoagulant therapy is indicated.

5. A combination product as claimed in any one of claims 1 to 4, wherein the derivative of melagatran is a prodrug of melagatran.

6. A combination product as claimed in claim 5, wherein the prodrug is of the formula
R¹O₂C—CH₂—(R)Cgl-Aze-Pab-OH,
wherein R¹ represents linear or branched C₁₋₆ alkyl and the OH group replaces one of the amidino hydrogens in Pab.

7. A combination product as claimed in claim 6, wherein R¹ represents methyl, ethyl, n-propyl, i-propyl or t-butyl.

8. A combination product as claimed in claim 6, wherein the prodrug is Glycine, N-[1-cyclohexyl-2-[2-[[[[4-[(hydroxyimino)aminomethyl]-phenyl]methyl]amino]carbonyl]-1-azetidinyl]-2-oxoethyl]-, ethyl ester, [S-(R*, S*)]-.

9. A combination product as claimed in any one of claims 1 to 7, which comprises Compound A or B or C or D (or pharmaceutically-acceptable salts thereof).

10. A method of making a kit of parts as defined in any one of claims 3 to 9, which method comprises bringing a component (a), as defined in any one of claims 3 to 9, into association with a component (b), as defined in any one of claims 3 to 9, thus rendering the two components suitable for administration in conjunction with each other.

11. A kit of parts comprising:
(I) one of components (a) and (b) as defined in any one of claims 3 to 9; together with
(II) instructions to use that component in conjunction with the other of the two components.

12. A method of treatment arrhythmia, which comprises administration of a combination product as defined in any one of claims 1 to 9 or a kit of parts as defined in claim 11 to a patient suffering from, or susceptible to, such a condition.

13. The use of a combination product as defined in any one of claims 1 to 9 or a kit of parts as defined in claim 11 for the manufacture of a medicament for the treatment or prophylaxis of a condition where anticoagulant therapy is indicated.

14. The use of melagatran or a pharmaceutically-acceptable derivative thereof and (1) a compound as defined in claim 1 of WO 01/28992 or (2) a compound of claim 34 of WO 01/28992 or (3) Compound A or B or C or D (or pharmaceutically-acceptable salts thereof) for the manufacture of a medicament for the treatment or prophylaxis of a condition where anticoagulant therapy is indicated.
